(19) United States
(12) Patent Application Publication
(10) Pub. No.: US 2025/0381360 A1
(43) Pub. Date: Dec. 18, 2025

(54) DEVICE AND PROCESS FOR GENERATING AN INLET PRESSURE AT A CONTROL VALVE OF A VENTILATOR

(71) Applicant: Drägerwerk AG & Co. KGaA, Lübeck (DE)

(72) Inventors: Hans-Ullrich HANSMANN, Lübeck (DE); Christian BRENDLE, Lübeck (DE)

(21) Appl. No.: 19/235,912

(22) Filed: Jun. 12, 2025

(30) Foreign Application Priority Data
Jun. 17, 2024 (DE) 10 2024 116 894.3

(51) Int. Cl. A61M 16/20; A61M 16/00

(52) U.S. Cl. CPC A61M 16/203; A61M 16/0066; A61M 2205/3334; A61M 2205/3344; A61M 2205/3365

(57) ABSTRACT

A device (10) generates an inlet pressure at a control valve of a ventilator (30) and includes a blower (1), a control unit (2) and the control valve (3). The blower is configured to draw in ambient air through an inlet (31), compress and forward it to the control valve. The control unit is configured to change a speed (N) of the blower and to operate the blower at a first speed level (n1) and determine a triggering time (t0) at which the speed of the blower is temporarily changed from the first speed level to a second speed level (n2) within a same breathing cycle of the ventilator. The first speed level is lower than the second speed level. A process for generating an inlet pressure (P) at the control valve as well as to the ventilator including the device and a computer program product are provided.

### CROSS REFERENCE TO RELATED APPLICATIONS

This application claims the benefit of priority under 35 U.S.C. § 119 of German Application 10 2024 116 894.3, filed Jun. 17, 2024, the entire contents of which are incorporated herein by reference.

### TECHNICAL FIELD

The present invention relates to a device and a process for generating an inlet pressure (upstream pressure/pre-pressure) at a control valve of a ventilator. The invention further relates to a ventilator with such a device and to a computer program product comprising instructions that cause a control unit to execute such a process.

### BACKGROUND

Ventilators are used to support or take over a patient's breathing. The aim is to supply the patient with sufficient oxygen and to promote the removal of carbon dioxide from the lungs. The patient is supplied with a flow of breathing gas (respiratory gas) whose properties, in particular pressure, volume flow and oxygen concentration, can be changed by the ventilator.

A ventilator usually includes a control valve with which the properties of a breathing gas flow, in particular pressure and volume flow, can be adjusted as required to supply the patient. The breathing gas flow usually comprises air, which can be obtained from a central gas supply of a hospital. Furthermore, ventilators are known that alternatively use air from the environment to form the breathing gas flow. The ventilator comprises a blower (fan) that draws in ambient air, compresses it and forwards (passes) it on to a patient interface via the control valve, wherein the patient interface comprises a breathing mask, for example. In addition, oxygen can be added to the ambient air, for example also from the central gas supply or from a gas cylinder, if this is required for ventilating the patient.

The blower comprises a motor that can usually be controlled by a control unit of the ventilator, wherein the speed of the motor or the blower can be adjusted. The speed corresponds to the number of revolutions per time. The pressure generated by the blower is essentially dependent on the speed of the blower. When the ambient air is forwarded or conveyed through the blower and the control valve to the patient interface, i.e. when a mass flow is generated, the pressure at the outlet of the blower drops, in particular due to restrictions in the flow path. In this case, the blower should be readjusted or regulated to maintain the pressure.

Essentially, two alternative operating variants of blowers are common in practice, namely dynamically operated blowers and statically operated blowers. A prerequisite for a dynamically operated blower is that the blower has a fast response behavior. For example, a dynamically operated blower is able to generate a pressure increase of 50 mbar within 100 ms. Accordingly, a dynamically operated blower must have a motor that is capable of achieving a corresponding increase in speed in a short time. Such a dynamically operated blower enables a flexible change in pressure, in particular an almost continuous adjustment of the speed and thus the pressure, over an entire breathing cycle of the ventilator.

Blowers that are operated statically have a much slower response time compared to a dynamically operated blower. For example, statically operated blowers require around 200 ms to raise the pressure by up to 50 mbar. This is generally too slow for flexible adjustment of the pressure of the breathing gas flow. It is therefore usually necessary to control the blower at a fixed speed during the entire breathing cycle so that a corresponding pressure is generated by the statically operated blower during the breathing cycle, particularly during an inspiratory phase.

In contrast to a dynamically operated blower, a statically operated blower has the advantage that it is more cost-effective to use. On the one hand, the manufacturing costs are lower, and on the other hand, the wear and the energy required are lower.

One problem with the use of statically operated blowers is that the pressure in the ventilation system drops due to the changing flow parameters during ventilation, in particular the volume flow or when a mass flow is generated when the control valve is opened, and the statically operated blower does not compensate for such pressure fluctuations. As a result, optimal supply of a patient with a demand-based breathing gas flow and corresponding pressure is not possible or only possible to a limited extent.

In practice, this problem is solved by the fact that the speed at which the statically operated blower is controlled, i.e. the pressure generated by the statically operated blower, is higher than the pressure required for the breathing gas flow to be delivered and set on the ventilator. As a result, the pressure generated by the statically operated blower controlled in this way is high enough that the pressure set by the operator on the ventilator can be reached while the breathing gas flow is being delivered to the patient through the control valve and has no or only insignificant pressure fluctuations, i.e. remains essentially constant.

The difference between the set pressure and the higher pressure provided by the statically operated blower is a so-called pressure control reserve. In this case, the pressure generated by the statically operated blower is therefore higher than the pressure set on the ventilator. The control valve arranged behind the blower in the direction of flow ensures that the pressure of the breathing gas flow corresponds to the set pressure.

The disadvantage of the pressure control reserve is the additional energy required to generate the higher pressure. Furthermore, the statically operated blower generates a louder operating noise when generating this higher pressure at a higher speed, which can be disturbing for the patient, and is subject to greater wear. It is therefore advantageous to keep the pressure control reserve of a statically operated blower as low as possible.

In the prior art, a process is known from DE10023656C1 which adjusts the pressure control reserve following a breathing cycle. During or after delivery of the breathing gas flow to the patient, it is assessed whether the pressure provided by the statically operated blower was too high or too low for a breathing cycle on the basis of an opening state of an additional bypass valve, wherein the pressure control reserve and thus the speed of the statically operated blower for a next breathing cycle is adjusted in accordance with the assessment.

### SUMMARY

Based on the solutions known from the prior art and the problems described above, the invention is based on the task of creating a cost-effective and improved device for generating an inlet pressure at a control valve of a ventilator, wherein the inlet pressure should be essentially constant during a breathing cycle of the ventilator.

The above task is solved by a device for generating an inlet pressure at a control valve of a ventilator with features according to the invention, a process for generating an inlet pressure at a control valve of a ventilator with features according to the invention as well as a ventilator with features according to the invention and a computer program product with features according to the invention. Further details of the invention are given in the dependent claims, the description and the drawings. Features and details which are described in connection with the device also apply in connection with the process, the ventilator and the computer program product, so that reference is always made or rather can be made mutually with respect to the disclosure of the individual aspects of the invention.

The device according to the invention for generating an inlet pressure at a control valve of a ventilator comprises a blower, a control unit and the control valve. The blower is configured to draw in (suck in) ambient air through an inlet, compress the ambient air and forward it on to the control valve. The control unit is configured to change the speed of the blower and thus influence the pressure and the volume flow of the ambient air drawn in. The device according to the invention is characterized in that the control unit is also configured to operate the blower at a first speed level and to determine a triggering time, in particular by evaluating available data, at which the speed of the blower is temporarily changed from the first speed level to a second speed level within the same breathing cycle of the ventilator. The first speed level is lower than the second speed level.

As described above, ventilators are used to support or take over the work of breathing from a patient. Anesthesia devices can also provide such a function, wherein they comprise at least components of a ventilator. In the context of the invention, both ventilators and anesthesia devices with a ventilation function are to be understood by the term ventilator.

In a ventilator which has a device configured according to the invention, the blower, in particular a radial blower, is in fluid connection with the inlet of the ventilator and the control valve and its speed can be changed by the control unit. The control unit preferably changes the speed of the blower by adjusting a control voltage that is applied to the blower and is preferably proportional to the speed. It is also conceivable that the control unit transmits an analog or digital control signal to the blower by wire or wirelessly, wherein the control signal comprises information indicating the speed of the blower. The control unit is preferably configured as a microprocessor. It is also conceivable that the control unit is configured as an FPGA (Field Programmable Gate Array), as an ASIC (Application-Specific Integrated Circuit) or as a comparable component for data processing and control.

According to the invention, the blower can be operated statically, with the speed being constant at least temporarily during a breathing cycle of the ventilator. The breathing cycle of the ventilator comprises an inspiration phase and an expiration phase.

During the inspiratory phase, the ventilator directs a flow of breathing gas to the patient. The control valve opens at least partially and directs at least part of the ambient air drawn in by the blower to an outlet of the ventilator as a breathing gas flow. The outlet is preferably connected to a patient interface for supplying a patient with the breathing gas flow with a fluid connection.

During the expiratory phase, the control valve is at least partially closed and no or only a small proportion of the breathing gas flow is directed to the patient. During expiration, a small proportion of the breathing gas flow is usually generated at the ventilator outlet to achieve a so-called PEEP (positive end-expiratory pressure).

When the ventilator takes over the patient's work of breathing, the periods of the inspiratory and expiratory phases are usually predefined by the user of the ventilator and can be set on the ventilator. In contrast, when the ventilator supports the patient's work of breathing, the periods of the inspiratory and expiratory phases are usually dependent on the patient's breathing effort (breathing work/respiratory effort), which can be detected by the ventilator. The breathing effort, i.e. an at least partial inhalation and/or exhalation of the patient, can be detected by means of suitable sensors of the ventilator.

The control unit is configured to determine a triggering time. The triggering time is preferably the time within a breathing cycle at or shortly before the time at which the control valve at least partially opens and provides a breathing gas flow, i.e. an inspiratory phase or conveying phase (delivery phase) of the ventilator during a breathing cycle. Preferably, the triggering time is a short time, for example in the range of 100 to 500 ms, before the start of the conveying phase. It is particularly preferred that the triggering time is up to one second before the conveying phase. In particular, the triggering time is before the conveying phase in such a way that the blower actually reaches the second speed level at least at the start of the conveying phase.

The triggering time is preferably derived from at least one setting of the ventilator and/or can be determined by monitoring the patient's breathing effort using suitable sensors (such as pressure sensors and/or volume flow sensor) on (or operatively connected to) the ventilator and/or on (or operatively connected to) the control unit. Furthermore, the triggering time can preferably be determined from the patient's breathing effort or conveying phases of previous breathing cycles.

The control unit is also configured to operate the blower at a first speed level outside the conveying phase of a breathing cycle of the ventilator and to increase the speed of the blower suddenly to a second speed level at the time of triggering. The second speed level is preferably at least indirectly adjustable on the ventilator and can therefore be predefined and/or determined by the ventilator and/or the control unit.

As described above, the pressure provided by the blower, i.e. the inlet pressure, drops when the control valve opens at least partially, i.e. in particular during the conveying phase when a mass flow is present. A drop in pressure means that less ambient air per time can be forwarded from the blower to the control valve, as the maximum flow rate that can be conveyed decreases and it takes longer for a required volume of breathing gas to be provided. As a result, a patient may not be optimally supplied with a breathing gas flow or not supplied as intended.

Advantageously, increasing the speed of the blower to a second speed level within a breathing cycle means that a pressure of the breathing gas flow set on the ventilator can be maintained, as the inlet pressure at the control valve, i.e. the pressure provided by the blower, is briefly increased at the outlet of the blower at least partially during the conveying phase in accordance with the second speed level and does not fall below the set pressure even when the inlet pressure falls due to the opening of the control valve during the conveying phase. This means that an essentially constant pressure in the flow direction downstream of the control valve, i.e. the pressure of the breathing gas flow, can be achieved.

The control unit is configured to change the speed of the blower temporarily within a breathing cycle, wherein the speed can be raised from a first speed level to a second speed level and can be lowered again during or directly after the conveying phase. Preferably, the lowering of the speed from the second speed level takes place after the conveying phase, wherein this essentially corresponds to the start of an expiratory phase of the breathing cycle of the ventilator. It is particularly preferable for the speed to be lowered from the second speed level after the start and before the end of the conveying phase, preferably up to two seconds, particularly preferably up to one second after the start of the conveying phase. It must be ensured that the second speed level is maintained long enough so that a pressure set on the ventilator can be maintained. Accordingly, the pressure control reserve can be raised within a breathing cycle for a limited period of time and at least during the conveying phase.

The temporary increase in speed within a breathing cycle of the blower has the particular advantage that the higher second speed level is only applied for a limited period of time during the breathing cycle and therefore less energy is required to operate the blower over the entire breathing cycle. Due to the increase in speed within a breathing cycle, a drop in the pressure of a breathing gas flow to be provided during this breathing cycle can be avoided in an advantageous way, thus enabling a patient to be supplied as required.

It is also advantageous that the lower first speed level during the breathing cycle results in lower operating noise, lower energy consumption and less wear on the blower. At the same time, the pressure of the breathing gas flow remains essentially constant during the conveying phase.

The blower has the advantage that it is inexpensive to manufacture and can be operated in an energy-saving manner, as no rapid changes in speed are required and the speed is only increased for a short time. Preferably, the blower is suitable for achieving a pressure increase of 50 mbar (equivalent to 5000 Pa) within a time range of 200 ms to 400 ms. Particularly preferably, the blower is suitable for achieving a pressure increase of 10 mbar (corresponds to 1000 Pa) within a time range of 200 ms to 400 ms. The pressure that can be generated by the blower is proportional to the speed of the blower.

According to a preferred embodiment of the device, the control unit is configured to change the speed of the blower from the second speed level to the first speed level during or following a conveying phase of the ventilator. As described above, the conveying phase is the period of time within a breathing cycle in which a breathing gas flow is provided at the outlet that is greater than the PEEP (Positive End-Expiratory Pressure). The conveying phase corresponds to the inspiratory phase when ventilating a patient.

Due to the lower first speed level compared to the second speed level, the blower is comparatively quiet over long operating times and is subject to less wear. Furthermore, less energy is required to operate the blower, which is a significant advantage for battery-powered ventilators in particular, as such a device can be operated for longer without an external power supply.

In a preferred embodiment of the device, the control unit is configured to determine the first speed level and the second speed level on the basis of a predefined pressure value and/or a predefined volume flow rate value. At least the second speed level is above a speed of the blower which is equivalent to the predefined pressure value, so that a drop in pressure during the conveying phase can be compensated. The predefined pressure value and/or the predefined flow rate value are preferably the settings on the ventilator that determine the properties of the breathing gas flow to be provided.

Preferably, the control unit determines the second speed level in such a way that the pressure of the breathing gas flow during the conveying phase corresponds to the predefined pressure value. In doing so, it calculates the pressure drop that occurs with a breathing gas flow with the predefined volume flow during the conveying phase. The second pressure level can be determined from the sum of the determined pressure drop and the predefined pressure value

The control unit is also configured to determine the first speed level in such a way that it is lower than the second speed level by a predefined proportion. Preferably, the first speed level is 5% to 15%, particularly preferably 15% to 20% below the second speed level. Furthermore, the first speed level is preferably lower than the second speed level by a predefined pressure constant, wherein the speed constant is dependent on the respective blower. Preferably, the pressure that can be achieved by the first speed level is in the range of 5 mbar to 30 mbar (corresponds to 500 Pa to 3000 Pa) below the pressure that can be achieved by the second speed level.

In an advantageous way, the first and second speed levels can be determined as a function of the predefined properties of the breathing gas flow, so that corresponding settings on the ventilator are taken into account. This means that the first and second speed levels can also be adapted to changes in the corresponding settings on the ventilator.

According to a preferred embodiment of the device, the control unit is configured to determine the triggering time during operation of the ventilator in a first operating mode on the basis of at least one ventilation parameter of the ventilator. The first operating mode comprises a functionality of the ventilator for mandatory or controlled ventilation, wherein the ventilator takes over the breathing effort of the patient.

In this first operating mode, the breathing cycle and its inspiration and expiration phases can be set on the ventilator. The ventilation parameters respiratory rate, duration of the breathing cycle, inspiration time and/or I:E ratio (quotient of inspiration to expiration time) are particularly relevant. The time and duration of the inspiratory phase within a breathing cycle are therefore predefined.

The control unit is configured to determine the triggering time at the latest at the time of the predefined inspiration phase, preferably earlier, and to change the speed of the blower to the second speed level.

In an advantageous way, the triggering time in the first operating mode of the ventilator can be adapted to its settings for the breathing cycle, ensuring that the speed can be changed at the time of the inspiratory phase or conveying phase.

In a preferred embodiment of the device, the control unit is configured to determine the triggering time during operation of the ventilator in a second operating mode on the basis of a time of a previous conveying phase of the ventilator. The second operating mode comprises a functionality of the ventilator to support the patient's breathing effort, wherein the ventilator determines the inspiration and expiration phase of a breathing cycle depending on a breathing effort of the patient detected by the ventilator. Such breathing effort is in particular self-breathing or spontaneous breathing of the patient

Preferably, the control unit is configured to operate the blower at the second speed level as long as no triggering time has been determined and/or is available, and to operate the blower initially at the first speed level as soon as a triggering time has been determined.

In this preferred embodiment, the triggering time can be determined by taking into account the times of previous conveying phases. The time gap between two previous conveying phases can be measured by the control unit and a time gap (time interval) between two conveying phases can be determined. The triggering time is determined on the basis of the time gap, wherein the triggering time corresponds to the time of the start of the previous conveying phase delayed by the time gap.

Preferably, several time gaps can be determined over a number of breathing cycles and an average value can be calculated from these, wherein the triggering time results from the time of the start of the previous conveying phase delayed by this average value.

Furthermore, the control unit is preferably configured to increment an expectation parameter (a counter) in an expectation time range by a constant value and to determine the triggering time, wherein the triggering time is reached as soon as the expectation parameter has reached a triggering threshold value.

The expectation time range is the time range from the start of a particular breathing cycle to the triggering time. At the beginning of the expectation time range, the expectation parameter can be incremented from zero. Preferably, the expectation parameter can be incremented by the value one per second.

If the expectation parameter reaches or exceeds the trigger threshold value, the trigger point is reached and the speed of the blower can be increased to the second speed level. Preferably, the trigger threshold value is predefined and is preferably set to 3 when the expectation parameter is incremented by one per second. The trigger threshold of 3 is an example for a default value, as the general breathing interval is about 3 seconds and in this example, the expectation parameter is incremented each second. The expectation parameter could instead be incremented much faster with a much higher trigger threshold value.

The control unit is particularly preferably configured to increment the expectation parameter up to the start of a conveying phase of the ventilator and to adjust the trigger threshold value to the expectation parameter when a deviation from the trigger threshold value is detected.

If the expectation parameter is greater than the trigger threshold value from the start of the conveying phase, the trigger threshold value is reduced by a predefined value by the control unit.

If the expectation parameter from the start of the conveying phase is smaller than the trigger threshold value, the trigger threshold value is increased by the control unit by a predefined value.

If the expectation parameter essentially matches the trigger threshold value from the start of the conveying phase, the trigger threshold value remains.

This solves the challenging problem where the timing of the conveying phase is unknown, which typically occurs when the patient is breathing spontaneously rather than controlled by the ventilator at given intervals.

In the case of the patient breathing spontaneously, the speed level of the blower needs to be increased before the unknown start time of the conveying phase to prevent a pressure drop that effects the breathing air properties during the conveying phase. The control unit is informed at what time a conveying phase occurs or is equipped to determine this start time of the conveying phase, i.e. for example in case the patient spontaneously breathes.

From the start of the conveying phase, the expectation parameter is incremented. When this value reaches the trigger threshold value, the speed level of the blower will be increased and the expectation parameter continues to increment until the actual start of the conveying phase. If the expectation parameter at the start of the conveying (pumping) phase deviates from the trigger threshold value, the trigger threshold value is adjusted—either increased or decreased—so that the next increase of the speed level of the blower, when the trigger threshold value is reached, occurs closer to the next expected conveying phase (while the patient breathes spontaneously/by himself/herself).

In an advantageous way, the triggering time can also be determined in an operating mode of the ventilator with supportive ventilation function. It is particularly advantageous to adjust the trigger threshold value, wherein the triggering time is at least as close as possible to the start of the conveying phase of the ventilator, so that the blower is only operated at the higher second speed level when it is necessary to avoid a drop in the pressure of the breathing gas flow.

The invention also relates to a ventilator with a device according to the invention or a device according to one of the embodiments described above. Due to the only temporarily increased speed of the blower of the device, the ventilator according to the invention requires less energy for operation, which is particularly advantageous for battery-operated ventilators, which thus offer a longer operating time. A further advantage is the comparatively low operating noise, which is at least less disturbing for a patient.

Furthermore, the invention relates to a process for generating an inlet pressure at a control valve of a ventilator, the process being suitable for being carried out by an embodiment of the devices described above and the ventilator according to the invention, in particular by the control unit. The process according to the invention comprises the following steps:
Receiving a pressure value and/or a volume flow rate value,
Determining a first speed level and a second speed level on the basis of the pressure value and/or the volume flow rate value, the first speed level being lower than the second speed level,
Controlling the blower with a control signal according to the first speed level,
Determining a triggering time,
Activating the blower with a control signal according to the second speed level at the time of triggering and with a time delay and within the same breathing cycle of the ventilator,
Actuating the blower with a control signal so that a speed results that is lower than the second speed level.

First, a pressure value and/or a volume flow rate value is received, preferably a specification from a user of the ventilator. According to the specification, a first and a second speed level are determined to control the blower. At the second speed level, an inlet pressure can be achieved at the inlet of the control valve downstream of the blower in the direction of flow that is higher than the pressure to be provided by the control valve according to the specification. This results in a pressure control reserve that enables the control valve to generate a demand-based breathing gas flow at the outlet of the control valve. The first speed level is preferably set to a value in the range of 5% to 20% lower than the second speed level, so that the blower preferably reaches the second speed level within 200 ms to 400 ms from the first speed level.

In a further step, the blower is controlled in such a way that its speed corresponds to the first speed level. Furthermore, a triggering time is determined at which the blower is controlled in such a way that its speed changes to the second speed level.

If the breathing cycle of the ventilator is predefined, the triggering time is preferably shortly before the conveying phase. It is particularly preferable for the triggering time to be up to one second before the conveying phase, so that it is ensured that the second speed level is present at the blower at the start of the conveying phase.

If, however, the ventilator is in a different operating mode and the breathing cycle is not predefined, for example, the triggering time is determined on the basis of at least one previous conveying phase of the ventilator. Preferably, the triggering time for subsequent breathing cycles of the ventilator is determined accordingly and adjusted if necessary.

Furthermore, the speed of the blower is lowered from the second speed level within a breathing cycle of the ventilator; the speed is preferably lowered to the first speed level. The time at which the speed is lowered preferably corresponds to the expiratory phase of the breathing cycle of the ventilator, which is predefined by a user or determined by the ventilator using suitable sensors. It is also conceivable that the speed is lowered after a predefined time following the triggering time, wherein the predefined time is preferably in the range of one to three seconds

Moreover, the invention relates to a computer program non-transitory, machine-readable, tangible data storage medium having stored thereon a computer program comprising instructions which, when executed by a control unit, cause the control unit to execute a process for generating an inlet pressure at a control valve of a ventilator. The ventilator comprises a device with the control valve, the control unit and a blower, wherein the blower draws in ambient air through an inlet, compresses it and forwards it on to the control valve. The control unit comprises a computer, a processor and/or a programmable hardware component and performs the following steps according to the computer program product:
Receiving a pressure value and/or a volume flow rate value,
Determining a first speed level and a second speed level on the basis of the pressure value and/or the volume flow rate value, the first speed level being lower than the second speed level,
Controlling the blower with a control signal according to the first speed level,
Determining a triggering time,
Activating the blower with a control signal according to the second speed level at the time of triggering and with a time delay and within the same breathing cycle of the ventilator,
Actuating the blower with a control signal so that a speed results that is lower than the second speed level.

Further features, tasks and effects of the invention can be seen from the following description of specific embodiments and the accompanying figures. Embodiments of the invention are described without limiting the general idea of the invention. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of a device as well as a course of a speed of the blower and a course of a volume flow of a breathing gas flow;

FIG. 2 is a schematic representation of time courses of a rotational speed, an inlet pressure and a volume flow; and

FIG. 3 is a simplified schematic representation of a ventilator with the device.

### DESCRIPTION OF PREFERRED EMBODIMENTS

Embodiments of the invention are described in detail below with reference to the attached figures.

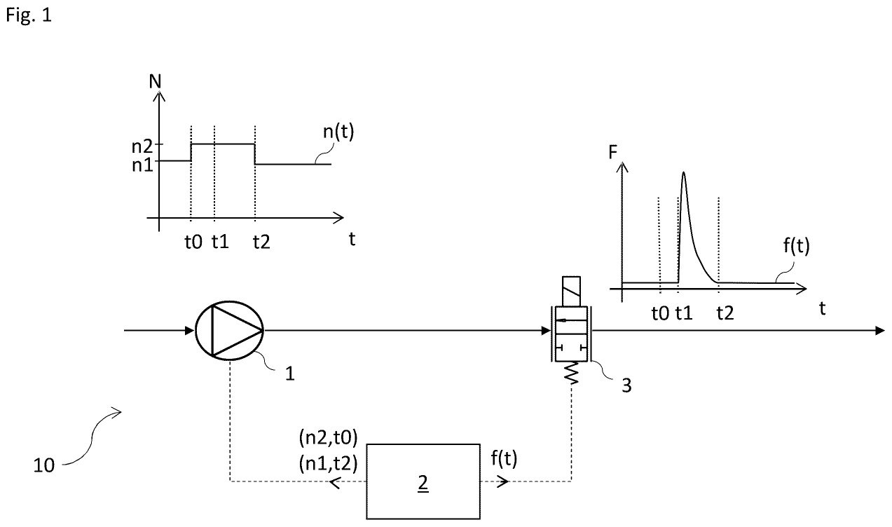

FIG. 1 shows a preferred embodiment of a device 10 with a blower 1 and a control valve 3 arranged downstream of the blower 1 in the direction of flow (the direction of flow is shown by continuous arrows). The blower 1 and the control valve 3 are in fluid connection. Furthermore, the blower 1 and the control valve 3 are electronically connected to a control unit 2, wherein the control unit 2 is configured to change at least the speed N of the blower 1 and the valve opening of the control valve 3 (signal and/or transmission paths are shown as a dashed line).

The control unit 2 is configured to initially operate the blower 1 at a first speed level n1, as shown in the diagram in FIG. 1 for the speed n(t) of the blower 1 over time. At a speed N greater than zero of the blower 1, the blower 1 draws in ambient air, compresses it and forwards it on to the control valve 3. If the control valve 3 is at least partially open, a mass flow with a volume flow F is created downstream of the control valve 3, which is dependent on the respective valve position.

Furthermore, the control unit 2 is configured to determine a triggering time t0 and to operate the blower 1 at a second speed level n2 at the triggering time t0, wherein the second speed level n2 is greater than the first speed level n1. In this embodiment example, the start of the conveying phase t1 is known and predefined by a user. The conveying phase t1 specifies the time at which the control unit 2 controls the control valve 3 in such a way that a mass flow is created that is suitable as a breathing gas flow for supplying a patient during an inspiratory phase of a breathing cycle. Based on the start of the conveying phase t1, the triggering time t0 can be determined by the control unit. In this embodiment example, the triggering time t0 is one second before the start of the conveying phase t1.

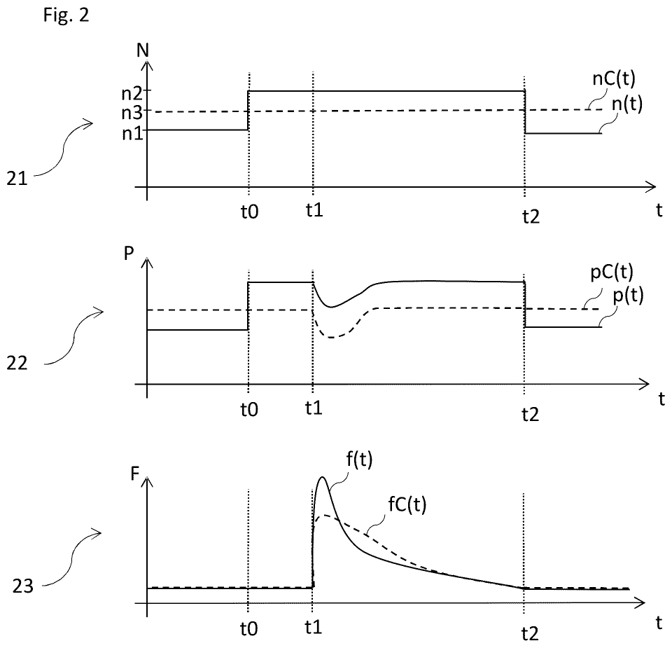

At the triggering time t0, i.e. one second before the start of the conveying phase t1, the control unit 2 changes the speed N of the blower 1 to the second speed level n2. As the speed N increases, the inlet pressure P at the inlet of the control valve 3 increases. Due to the second speed level n2 of the blower 1 and the resulting increased inlet pressure P, it is advantageously ensured that a drop in the inlet pressure P at the start of the conveying phase t1 has little or no effect on the volume flow F of the breathing gas flow, wherein the breathing gas flow is supplied to the patient and is shown in the volume flow time course f(t). The relationship between speed N, inlet pressure P and volume flow F is explained in more detail in the description of FIG. 2.

The control unit 2 is also configured to operate the blower 1 again at a lower speed level than the second speed level n2 at a reduction time t2, which temporally follows the start of the conveying phase. In this embodiment, the second speed level can be lowered to the first speed level n1 at the reduction time. In this embodiment example, the reduction time essentially corresponds to a predefined expiration phase of a breathing cycle. It is also conceivable that the reduction time t2 is before the start of the expiration phase. For example, the reduction time t2 is up to one second after the start of the conveying phase.

By temporarily changing the speed N, the device 10 can be used in an energy-saving manner, as the blower 1 is only operated at an increased speed N, namely the second speed level n2, for a short time. This also results in less noise pollution and less wear on the blower 1.

FIG. 2 shows a schematic representation of the time courses of various parameters of a device 10. The following time courses are shown: speed N of a blower 1, inlet pressure P, which is applied to a control valve 3, and volume flow F downstream of the control valve 3. In the following, the dependency between speed N, inlet pressure P and volume flow F will be described in more detail, wherein the time courses 21, 22, 23 represent at least partially overlapping time ranges, which comprise a triggering time t0, a start of the conveying phase t1 and a reduction time t2. Furthermore, FIG. 2 shows a comparison of a speed time course n(t) according to one embodiment of the invention with a time course with constant speed nC(t) of a blower as well as the resulting time courses p(t), pC(t), f(t), fC(t).

First, the effect of a constant speed level n3 of a blower as shown in FIG. 2 is described, as is to be expected with devices known from the state of the art. A speed time course nC(t) with constant speed n3 over the time t leads to an essentially constant inlet pressure P up to the start of the conveying phase t1. Due to the increased volume flow F at the start of the conveying phase t1, the inlet pressure P decreases, as shown in the inlet pressure course 22 and the inlet pressure time course at constant speed pC(t). In this case, the drop in the inlet pressure P means that the maximum possible volume flow F is lower than with a higher inlet pressure P. This results in a flatter course of the volume flow fC(t), as shown in the volume flow course 23.

In contrast to the flat time course of the volume flow at constant speed fC(t), a temporary change in the speed N of the blower 1 according to the invention results in a steeper time course of the volume flow f(t) at the triggering time t0, as shown in the volume flow course 23. Although the inlet pressure P also decreases, as shown in the inlet pressure course pC(t), 22, the inlet pressure P is still high enough to achieve a comparatively larger maximum volume flow F according to f(t). In an advantageous way, a predefined volume of a mass flow or breathing gas flow is thus available in a shorter time.

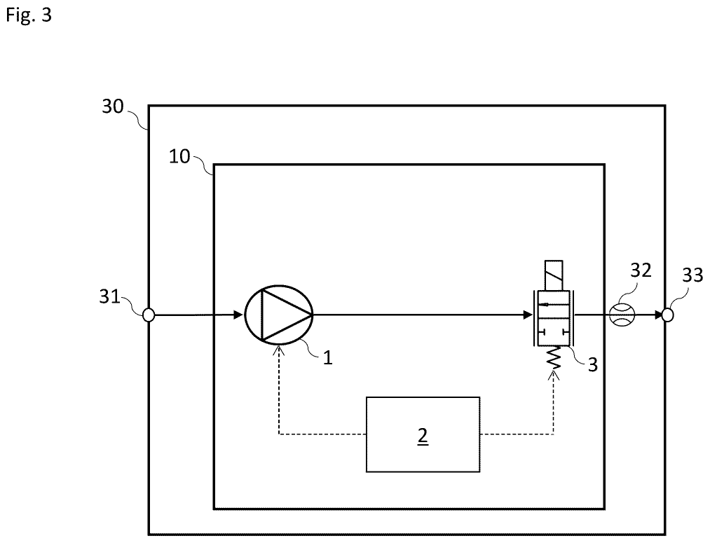

FIG. 3 shows a preferred embodiment example by means of a highly simplified, schematic representation of a ventilator 30 with a device 10 comprising a blower 1 and a control valve 3 arranged downstream of the blower, each of which is electronically connected to a control unit 2. Furthermore, the ventilator 30 comprises a volume flow sensor 32 as well as an inlet 1 and an outlet 7, wherein the inlet 31 is in fluid connection with the blower 1 in and the outlet 33 is in fluid connection with the control valve 3. The volume flow sensor 32 is arranged downstream of the control valve 3 and upstream of the outlet 33 and is used to measure the volume flow F of a breathing gas flow to be provided.

The control unit 2 is configured to set and change the speed N of the blower 1 and the valve position of the control valve 3. At a speed N greater than zero, the blower draws in ambient air through the inlet 31, compresses it and forwards it on to the control valve 3 so that an inlet pressure P is present at the control valve 3. As soon as the control valve 3 opens at least partially, a mass flow is created through the control valve 3, which can be used as a breathing gas flow to ventilate a patient not shown. The breathing gas flow leaves the control valve 3 in the direction of flow. The breathing gas flow is then available at outlet 33 and can be forwarded on to the patient via a patient interface not shown, for example a respiratory mask.

In this embodiment, the breathing cycle for ventilating a patient is predefined so that the times of a respective conveying phase and an expiration phase of the control unit 2 are known. Consequently, the times of an inspiratory phase and an expiratory phase of the breathing cycle are known. The inspiration phase essentially corresponds to the start of the conveying phase t1. Based on the start of the conveying phase t1, the control unit determines the triggering time t0, which indicates when the speed N of the blower 1 is raised from a first speed level n1 to a second speed level n2

At the triggering time t0, which is up to one second before the start of the conveying phase t1, the speed N of the fan is increased, resulting in a higher inlet pressure P at control valve 3. At the start of conveying phase t1, control valve 3 opens to generate a flow of breathing gas. Due to the higher inlet pressure, a predefined volume of the breathing gas flow is quickly available and ensures that a patient is ventilated as required in an advantageous manner.

While specific embodiments of the invention have been shown and described in detail to illustrate the application of the principles of the invention, it will be understood that the invention may be embodied otherwise without departing from such principles.

### LIST OF REFERENCE SYMBOLS

1 Blower
2 Control unit
3 Control valve
10 Device
21 Speed course
22 Pressure course
23 Volume flow course
30 Ventilator
31 Inlet
32 Volume flow sensor
33 Outlet
N Speed of the blower
n1 First speed level
n2 Second speed level
n3 Constant speed level
n(t) Speed time course
nC(t) Speed time course at constant speed
t Time
t0 Triggering time
t1 Start of the conveying phase
t2 Reduction time
F Volume flow of the breathing gas flow
f(t) Volume flow time course
fC(T) Volume flow rate time course at constant speed
P Inlet pressure
p(t) Inlet pressure time course
pC(t) Inlet pressure time course at constant speed

What is claimed is:

1. A device for generating a control valve inlet pressure for a ventilator, the device comprising:
a control valve;
a blower, which is configured to draw in ambient air through a ventilator inlet, to compress the ambient air and to forward the ambient air on to the control valve; and
a control unit, which is configured to change a rotational speed of the blower and to operate the blower at a first speed level and to determine a triggering time at which the speed of the blower is temporarily changed from the first speed level to a second speed level within a same breathing cycle of the ventilator, the first speed level being lower than the second speed level.

2. A device according to claim 1, wherein the triggering time is before a start of a conveying phase of the ventilator.

3. A device according to claim 1, wherein the control unit is configured to change the speed of the blower from the second speed level to the first speed level during or following a conveying phase of the ventilator.

4. A device according to claim 1, wherein the control unit is configured to determine the first speed level and to determine the second speed level based on a predefined pressure value and/or based on a predefined volume flow value.

5. A device according to claim 1, wherein the control unit is configured to determine the triggering time based on a ventilation parameter of the ventilator during an operation of the ventilator in an operating mode.

6. A device according to claim 1, wherein the control unit is configured to determine the triggering time based on a time of a previous conveying phase of the ventilator during operation of the ventilator in an operating mode.

7. A device according to claim 6, wherein the control unit is configured to increment an expectation parameter in an expectation time range by a constant value and to determine the triggering time, the triggering time being present as soon as the expectation parameter has reached a triggering threshold value.

8. A device according to claim 7, wherein the control unit is configured to increment the expectation parameter up to the start of a conveying phase of the ventilator and to adjust the trigger threshold value to the expectation parameter when a deviation from the trigger threshold value is detected.

9. A device according to claim 1, in combination with the ventilator inlet and a ventilator outlet, the combination forming a ventilator.

10. A device according to claim 9, wherein the ventilator further comprises a volume flow sensor arranged downstream of the control valve and upstream of the ventilator outlet.

11. A process for generating an inlet pressure at a control valve of a ventilator, the ventilator comprising a control unit and a blower, wherein the blower draws in ambient air through an inlet, compresses ambient air and forwards ambient air on to the control valve, wherein the control unit carries out the steps of:
receiving a pressure value and/or a volume flow rate value;
determining a first speed level and determining a second speed level based on the pressure value and/or the volume flow rate value, the first speed level being lower than the second speed level;
controlling the blower with a control signal according to the first speed level;
determining a triggering time;
controlling the blower with a control signal according to the second speed level at the triggering time; and
time-delayed from the triggering time and within a same breathing cycle of the ventilator, controlling the blower with a control signal such that a speed is obtained which is lower than the second speed level.

12. A process according to claim 11, wherein the triggering time is before a start of a conveying phase of the ventilator.

13. A process according to claim 11, wherein the lowering of the speed from the second speed level takes place during a conveying phase of the ventilator or following the conveying phase of the ventilator.

14. A computer program non-transitory, machine-readable, tangible data storage medium having stored thereon a computer program comprising instructions which, when executed by a control unit, cause the control unit to execute a process for generating an inlet pressure at a control valve of a ventilator comprising a device with the control valve, the control unit and a blower, wherein the blower draws in ambient air through a ventilator inlet, compresses ambient air and forwards the ambient air on to the control valve, the instructions execution comprising the steps of:
receiving a pressure value and/or a volume flow rate value;
determining a first speed level and determining a second speed level based on the pressure value and/or the volume flow rate value, the first speed level being lower than the second speed level;
controlling the blower with a control signal according to the first speed level;
determining a triggering time;
controlling the blower with a control signal according to the second speed level at the triggering time; and
with a time-delay from the time of triggering and within a same breathing cycle of the ventilator, controlling the blower with a control signal so that a speed is obtained which is lower than the second speed level.

15. A computer program tangible data storage medium according to claim 14, wherein the triggering time is before a start of a conveying phase of the ventilator.

16. A computer program tangible data storage medium according to claim 14, wherein the lowering of the speed from the second speed level takes place during or following a conveying phase of the ventilator.
